(19) United States
(12) Patent Application Publication
(10) Pub. No.: US 2026/0301924 A1
(43) Pub. Date: Oct. 1, 2026

(54) Methods, Systems, and Devices for Interpreting Non-Human Patient Medical Images

(71) Applicant: IDEXX LABORATORIES, INC., Westbrook, ME (US)

(72) Inventors: Katherine Healy, Portland, ME (US); Benjamin Schestopol, Scarborough, ME (US); Rebecca Baumruck, Westbrook, ME (US); Pavan Durgempudi, Portland, ME (US); Allison Spake, Westbrook, ME (US); Christian Cortner, Westbrook, ME (US); Patrick Doyle, Westbrook, ME (US); Michael Bailey, Westbrook, ME (US); Caitlin Robar, Westbrook, ME (US); Charlie White, Westbrook, ME (US)

(21) Appl. No.: 19/629,631

(22) Filed: Mar. 26, 2026

Related U.S. Application Data
(60) Provisional application No. 63/779,160, filed on Mar. 27, 2025.

(51) Int. Cl. G16H 30/40; G16H 10/60; G16H 50/20; G16H 50/30

(52) U.S. Cl. CPC G16H 30/40; G16H 10/60; G16H 50/20; G16H 50/30

(57) ABSTRACT

A method includes receiving, by a computing device, one or more medical images of a non-human patient. The method includes providing the one or more medical images to a machine learning model. The method includes receiving, from the machine learning model, a medical interpretation associated with the one or more medical images. The medical interpretation is based on the machine learning model (i) identifying a medical condition based on the one or more medical images and (ii) determining a confidence score that the medical condition is applicable to the non-human patient. The method includes transmitting the one or more medical images and the medical interpretation to a second computing device. At least one of the confidence score threshold or machine learning logic associated with the machine learning model is updated based on feedback from the second computing.

### CROSS-REFERENCE TO RELATED APPLICATIONS

This application claims the benefit of co-pending U.S. Provisional Patent Application Serial No. 63/779,160, filed Mar. 27, 2025, which is hereby incorporated by reference in its entirety.

### FIELD OF THE DISCLOSURE

The present disclosure involves systems and methods for analyzing, annotating, and interpreting non-human patient medical images using machine learning.

### BACKGROUND

When treating non-human patients (e.g., dogs, cats, birds, etc.), veterinarians may sometimes use medical imaging technology to diagnose medical conditions. For example, a non-human patient may undergo a radiology examination such that one or more medical images (e.g., radiological images) of the non-human patient are captured. These medical images may be transmitted to a specialist (e.g., a radiologist) for interpretation and used to form all or part of a specific medical treatment plan and/or protocol for a non-human patient.

In some scenarios, it may take the specialist a relatively long time to analyze and/or otherwise interpret theses medical images and identify the medical condition of the non-human patient. The length of time for interpreting the medical images may be based on different factors. As examples, the specialist may need to (i) study a medical history of the non-human patient, (ii) identify a species of the non-human patient, (iii) compare the medical images of the non-human patient to other medical images of the species, (iv) consider an age of the non-human patient, etc. In some examples, the length of time for analyzing and/or otherwise interpreting the medical images may be due to an extensive worklist of the specialist.

For example, the specialist may have other medical images to interpret prior to interpreting the medical images of the non-human patient. In any event, one or more deleterious effects may result from this unnecessary delay of medical analysis and treatment of the non-human patient for analyzing and/or otherwise interpreting these medical images.

### SUMMARY

In an example, a computer-implemented method for interpreting non-human patient medical images is described. In examples, the computer-implemented method includes receiving, by a computing device, one or more medical images of a non-human patient. In examples, the computer-implemented method includes providing, by the computing device, the one or more medical images to a machine learning model. In examples, the computer-implemented method includes receiving, by the computing device and from the machine learning model, a medical interpretation associated with the one or more medical images. In examples, the medical interpretation is based on the machine learning model (i) identifying a medical condition based on the one or more medical images and (ii) determining a confidence score that the medical condition is applicable to the non-human patient. In examples, the computer-implemented method includes determining, by the computing device, that the confidence score satisfies a confidence score threshold. In examples, the computer-implemented method includes transmitting, by the computing device, the one or more medical images and the medical interpretation to a second computing device in response to a determination that the confidence score satisfies the confidence score threshold. At least one of the confidence score threshold or machine learning logic associated with the machine learning model is updated based on feedback from the second computing device.

In another example, a computing device is described. In examples, the computing device includes one or more processor and a tangible, non-transitory computer-readable medium including instructions that, when executed by the one or more processors, cause the one or more processors to perform operations. In examples, the operations include receiving one or more medical images of a non-human patient. In examples, the operations include providing the one or more medical images to a machine learning model. In examples, the operations include receiving, from the machine learning model, a medical interpretation associated with the one or more medical images. In examples, the medical interpretation is based on the machine learning model (i) identifying a medical condition based on the one or more medical images and (ii) determining a confidence score that the medical condition is applicable to the non-human patient. In examples, the operations include determining that the confidence score satisfies a confidence score threshold. In examples, the operations include transmitting the one or more medical images and the medical interpretation to a second computing device in response to a determination that the confidence score satisfies the confidence score threshold. At least one of the confidence score threshold or machine learning logic associated with the machine learning model is updated based on feedback from the second computing device.

In another example, a tangible, non-transitory computer-readable medium is described. In examples, the tangible, non-transitory computer-readable medium includes instructions that, when executed by one or more processors of a computing device, cause the one or more processor to perform operations. In examples, the operations include receiving one or more medical images of a non-human patient. In examples, the operations include providing the one or more medical images to a machine learning model. In examples, the operations include receiving, from the machine learning model, a medical interpretation associated with the one or more medical images. In examples, the medical interpretation is based on the machine learning model (i) identifying a medical condition based on the one or more medical images and (ii) determining a confidence score that the medical condition is applicable to the non-human patient. In examples, the operations include determining that the confidence score satisfies a confidence score threshold. In examples, the operations include transmitting the one or more medical images and the medical interpretation to a second computing device in response to a determination that the confidence score satisfies the confidence score threshold. At least one of the confidence score threshold or machine learning logic associated with the machine learning model is updated based on feedback from the second computing device.

The features, functions, and advantages that have been discussed can be achieved independently in various examples or may be combined in yet other examples. Further details of the examples can be seen with reference to the following description and drawings.

### BRIEF DESCRIPTION OF THE FIGURES

The above, as well as additional features will be better understood through the following illustrative and non-limiting detailed description of example embodiments, with reference to the appended drawings.

FIG. 1 illustrates a diagram of an example system for interpreting non-human patient medical images using machine learning, according to an example embodiment.

FIG. 2 illustrates a diagram of an example process for interpreting non-human patient medical images using machine learning, according to an example embodiment.

FIG. 3 illustrate a diagram of an example process for training a machine learning model based on a radiologist-determined medical condition, according to an example embodiment.

FIG. 4 illustrates a diagram of an example an example process 400 for selectively sending a model-based interpretation of medical images to a radiologist, according to an example embodiment.

FIG. 5 illustrates a diagram of another example system for interpreting non-human patient medical images using machine learning, according to an example embodiment.

FIG. 6 illustrates a method, according to an example embodiment.

All the figures are schematic, not necessarily to scale, and generally only show parts which are necessary to elucidate example embodiments, wherein other parts may be omitted or merely suggested.

### DETAILED DESCRIPTION

Example embodiments will now be described more fully hereinafter with reference to the accompanying drawings. That which is encompassed by the claims may, however, be embodied in many different forms and should not be construed as limited to the embodiments set forth herein; rather, these embodiments are provided by way of example. Furthermore, like numbers refer to the same or similar elements or components throughout.

Within examples, the disclosure is directed to interpreting non-human patient medical images using machine learning to reduce a read time for a radiologist. To illustrate, medical images (e.g., radiology images, x-ray images, gamma ray images, etc.) of a non-human patient may be provided to a machine learning model that is trained and retrained to interpret medical images. In some examples, in addition to the medical images of the non-human patient, one or more medical indicators associated with the non-human patient may be provided to the machine learning model. In examples, the one or more medical indicators may include an age of the non-human patient, a weight of the non-human patient, a species of the non-human patient, a breed of the non-human patient, one or more symptoms of the non-human patient, a preexisting condition of the non-human patient, a geographical location of the non-human patient, etc. Based on the medical images, and optionally the medical indicators, the machine learning model may generate an interpretation (e.g., a reading) of the medical images. For example, the machine learning model may determine whether the non-human patient has a medical condition.

In addition to the medical images, the interpretation by the machine learning model may be selectively sent to a radiologist. For example, a confidence score of the medical condition determined by the machine learning model exceeds a threshold score, the interpretation may be sent to the radiologist. In examples, the radiologist may use the interpretation of the machine learning model as a starting point to interpret the medical images. For example, the radiologist can simply verify whether the interpretation by the machine learning model is accurate. If the radiologist determines that the interpretation by the machine learning model is accurate, the radiologist may experience significant time savings in interpreting the medical images. However, if the radiologist determines that the interpretation by the machine learning model is not accurate, the radiologist may provide feedback to the machine learning model to further train the machine learning model. Training the machine learning model using inaccurate machine learning model interpretations may increase the likelihood that future machine learning model interpretations are accurate, which will also result in significant time savings and/or improvement medical treatments for one or more non-human patients.

Accordingly, features of the present disclosure can help to address these and other issues to provide an improvement to select technical fields. More specifically, features of the present disclosure help address issues within and provide improvements for select technical fields, which include for example, veterinary and medical care communication systems, medical record and medical treatment protocol and triage processing systems, computer-based analysis systems, and graphical user interfaces (GUIs), all of which also help further improve medical treatments for one or more non-human patients. These features and more will now be described.

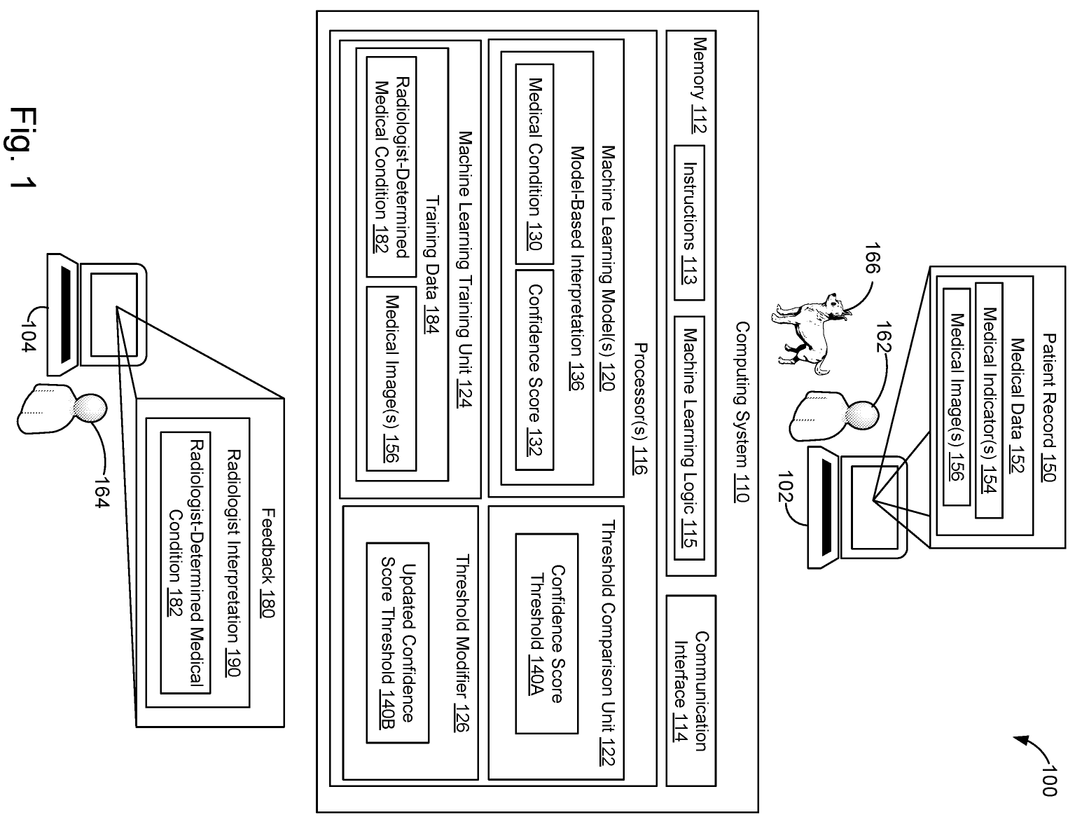

Particular implementations are described herein with reference to the figures. In the description, common features may be designated by common reference numbers throughout the figures. In some figures, multiple instances of a particular type of feature are used. Although these features are physically and/or logically distinct, the same reference number is used for each, and the different instances are distinguished by addition of a letter to the reference number. When the features as a group or a type are referred to herein (e.g., when no particular one of the features is being referenced), the reference number is used without a distinguishing letter. However, when one particular feature of multiple features of the same type is referred to herein, the reference number is used with the distinguishing letter. For example, referring to FIG. 1, confidence score thresholds are illustrated and associated with reference numbers 140A and 140B. When referring to a particular one of the confidence score thresholds, such as the confidence score threshold 140A, the distinguishing letter “A” is used. However, when referring to any one of the confidence score thresholds or to the confidence score thresholds as a group, the reference number 140 may be used without a distinguishing letter.

Referring now to the figures, FIG. 1 is a diagram of an example system 100 for interpreting non-human patient medical images using machine learning.

In examples, the system 100 includes a computing device 102, a computing device 104, and a computing system 110. In examples, the computing devices 102, 104 may be one of laptop computers, desktop computers, personal digital assistants (PDAs), tablets, workstation terminals, mobile phones, controllers, or any other devices that can receive, transmit, and display information, such as medical records. In one scenario, the computing device 102 may be accessible to a veterinarian 162, and the computing device 104 may be accessible to a radiologist 164.

As described below, the computing system 110 may be configured to interpret non-human patient medical images 156 using machine learning. For example, the computing system 110 may use machine learning logic 115 to generate a model-based interpretation 136 of the medical images 156 (e.g., radiology images of a non-human patient 166). Using the computing device 104, the radiologist 164 can access the medical images 156 and the corresponding model-based interpretation 136 of the medical images 156. In examples, the radiologist 164 can use the model-based interpretation 136 of the medical images 156 as a starting point to interpret the medical images 156 (e.g., to generate a radiologist interpretation 190). In examples, the radiologist interpretation 190 of the medical images 156 may be compared model-based interpretation 136 of the medical images 156 to generate feedback 180 that indicates whether the model-based interpretation 136 is applicable (e.g., whether the model-based interpretation is correct and/or accurate). Based on the feedback 180, the computing system 110 can update the machine learning logic 115 used to generate the model-based interpretation 136, perform other actions to improve an interpretation accuracy of the medical images 156, or a combination thereof.

In some examples, the computing system 110 may be integrated into the computing device 102. For example, the computing device 102 accessible to the veterinarian 162 (e.g., the party requesting interpretation of the medical images 156) may (i) use the machine learning logic 115 to interpret the medical images 156 and (ii) send the model-based interpretation 136 of the medical images 156 to the computing device 104 accessible to the radiologist 164 (e.g., the party interpreting the medical images 156). In some examples, the computing system 110 may be integrated into the computing device 104. For example, the computing device 104 accessible to radiologist 164 may (i) use the machine learning logic 115 to interpret the medical images 156 and (ii) display the model-based interpretation 136 of the medical images 156 for the radiologist 164. In some examples, the computing system 110 may be integrated into a server (not shown). For example, the medical images 156 from the computing device 102 accessible to the veterinarian 162 may be transmitted to the server, and the server may (i) use the machine learning logic 115 to interpret the medical images 156 and (ii) send the model-based interpretation 136 of the medical images 156 to the computing device 104 accessible to the radiologist 164.

In examples, the computing system 110 includes a memory 112, a communication interface 114, and one or more processors 116 (herein referred to interchangeably as “the processor(s) 116” or the processor 116). In examples, the memory 112 can include one or more volatile, non-volatile, removable, and/or non-removable storage components, such as magnetic, optical, or flash storage, and/or can be integrated in whole or in part with the processor(s) 116. Further, the memory 112 can take the form of a non-transitory computer-readable storage medium, having stored thereon program instructions 113 (e.g., compiled or non-compiled program logic and/or machine code) that, when executed by the processor(s) 116, cause the computing system 110 to perform one or more acts and/or functions, such as those described in this disclosure. As such, the computing system 110 can be configured to perform one or more acts and/or functions, such as those described in this disclosure. Such program instructions 113 can define and/or be part of a discrete software application. In some instances, the computing system 110 can execute program instructions 113 in response to receiving an input (e.g., a patient record 150, the medical images 156, and/or medical indicators 154) from the communication interface 114. The memory 112 can also store other types of data, such as those types described in this disclosure. For example, as described in greater detail below, the memory 112 may also store the machine learning logic 115 that is used by the processor 116 to interpret medical images 156.

In examples, the communication interface 114 can allow the computing system 110 to connect to and/or communicate with other entities, such as the computing devices 102, 104, according to one or more protocols. In one example, the communication interface 114 can be a wired interface, such as an Ethernet interface or a high-definition serial-digital-interface (HD-SDI). In another example, the communication interface 114 can be a wireless interface, such as a cellular or WI FI interface. In this disclosure, a connection can be a direct connection or an indirect connection, the latter being a connection that passes through and/or traverses one or more entities, such as such as a router, switcher, or other network device. Likewise, in this disclosure, a transmission can be a direct transmission or an indirect transmission.

In examples, the processor 116 can include a general-purpose processor (e.g., a microprocessor) and/or a special-purpose processor (e.g., a digital signal processor (DSP)). In examples, the processor 116 includes one or more machine learning models 120 (herein referred to as “the machine learning model 120”), a threshold comparison unit 122, a machine learning training unit 124, and a threshold modifier 126. In some examples, one or more components of the processor 116 may be implemented using dedicated hardware. To illustrate, in some examples, one or more components of the processor 116 may be implemented via an application-specific integrated circuit (ASIC) or a field-programmable gate array (FPGA) device. In some examples, one or more components of the processor 116 may be implemented using software or firmware. To illustrate, in some examples, operations performed by one or more of the components of the processor 116 can be implemented by executing the instructions 113 stored in the memory 112.

As referenced above, the computing system 110 may be configured to use the machine learning logic 115 to interpret non-human patient medical images 156. To illustrate, the computing system 110 may be configured to receive, via the communication interface 114, the one or more medical images 156 of the non-human patient 166. For example, the veterinarian 162 associated with the computing device 102 may send the patient record 150 to the computing system 110. In examples, the patient record 150 may include medical data 152 of the non-human patient 166 (e.g., a cat, a dog, a bird, etc.). As illustrated in FIG. 1, the medical images 156 and one or more medical indicators 154 may be part of medical data 152 that is included in the patient record 150 of the non-human patient 166.

In examples, the medical indicators 154 may include information stored in association with the one or more medical records associated with the non-human patient 166. As non-limiting examples, the one or more medical indicators may include an age of the non-human patient 166, a weight of the non-human patient 166, a species of the non-human patient 166, a breed of the non-human patient 166, one or more symptoms of the non-human patient 166, a preexisting condition of the non-human patient 166, a geographical location of the non-human patient 166, or a combination thereof.

In some examples, the one or more medical indicators 154 associated with the non-human patient 166 includes information stored in association with one or more medical records associated with another non-human patient 166 that shares one or more attributes with the non-human patient 166. In examples, the one or more attributes may include an age, a weight, a species, a breed, one or more symptoms, a preexisting condition, a geographical location, or a combination thereof.

In examples, the computing system 110 (e.g., the processor 116) may be configured to provide the medical images 156 to the machine learning model 120. In examples, the machine learning model 120 may be implemented by the processor 116 executing the machine learning logic 115 stored in the memory 112. Based at least on the medical images 156, the machine learning model 120 may be configured to identify a medical condition 130 associated with the non-human patient 166. In examples, the identified medical condition 130 may be based on a confidence score 132 that indicates a likelihood that the medical condition 130 is applicable to the non-human patient 166. In examples, the medical condition 130 and the confidence score 132 may be part of the model-based interpretation 136 of the medical images 156. For example, the model-based interpretation 136 of the medical images 156 may indicate the medical condition 130 of the non-human patient 166, as determined by the machine learning model 120.

In some examples, the computing system 110 (e.g., the processor 116) may be configured to provide one or more of the medical indicators 154 associated with the non-human patient 166 to the machine learning model 120. In these examples, the machine learning model 120 may identify the medical condition 130 and determine the confidence score 132 based on the medical indicators 154 and the medical images 156. Thus, the model-based interpretation 136 of the medical images 156 may be further based on the medical indicators 154 associated with the non-human patient 166.

In examples, the threshold comparison unit 122 may be configured to determine whether the confidence score 132 satisfies a confidence score threshold 140A. As described above, the confidence score 132 indicates a likelihood that the medical condition 130 is applicable to the non-human patient 166. If the confidence score 132 satisfies (e.g., is equal to or greater than) the confidence score threshold 140A, the processor 116 may determine that the medical condition 130 of the non-human patient 166, as determined by the machine learning model 120, is reliable. In one scenario, the computing system 1100 may send the model-based interpretation 136 of the medical images 156 to the computing device 104 based on the comparison between the confidence score 132 and the confidence score threshold 140A. In this scenario, the computing system 110 may send, via the communication interface 114, the patient record 150 (e.g., medical images 156) and the model-based interpretation 136 of the medical images 156 to the computing device 104 if the confidence score 132 satisfies the confidence score threshold 140A. Thus, the computing system 110 may be configured to transmit the medical images 156 and the model-based interpretation 136 of the medical images 156 (e.g., the medical condition 130) to the computing device 104 in response to determination that the confidence score 132 satisfies the confidence score threshold 140A.

However, if the confidence score 132 fails to satisfy the confidence score threshold 140A, the processor 116 may determine that the medical condition 130 of the non-human patient 166, as determined by the machine learning model 120, is not reliable. In this scenario, the computing system 110 may bypass sending model-based interpretation 136 of the medical images 156 to the computing device 104 and may only send the patient record 150 (e.g., the medical images 156) to the computing device 104.

In other scenarios, the computing system 110 may send the model-based interpretation 136 of the medical images 156 to the computing device 104 regardless of whether the confidence score 132 satisfies the confidence score threshold 140A. As described in greater detail below, in scenarios where the medical condition 130, as determined by the machine learning model 120, is not applicable to the non-human patient 166, the radiologist 164 may provide corresponding feedback 180 that can be used to further train the machine learning model 120 (e.g., update the machine learning logic 115).

In examples, the radiologist 164 may perform a radiologist interpretation 190 of the medical images 156 to determine a radiologist-determined medical condition 182 of the non-human patient 166. As illustrated in FIG. 1, the radiologist interpretation 190 of the medical images 156 may indicate the radiologist-determined medical condition 182 of the non-human patient 166. Upon determining the radiologist-determined medical condition 182 of the non-human patient 166, the radiologist 164 may determine whether the model-based interpretation 136 is consistent with the radiologist interpretation 190. For example, the radiologist 164 may compare the radiologist-determined medical condition 182 to the medical condition 130, as determined by the machine learning model 120. Based on the comparison, the radiologist 164 may provide feedback 180 to the computing system 110. In examples, the feedback 180 may indicate whether the medical condition 130, as determined by the machine learning model 120, is applicable to the non-human patient 166.

In response to the computing system 110 receiving the feedback 180, the machine learning training unit 124 may be configured to generate training data 184. In examples, the training data 184 may be generated based on the medical images 156 and the feedback 180 (e.g., the radiologist-determined medical condition 182). In examples, the machine learning training unit 124 may use the training data 184 to update the machine learning logic 115. For example, the training data 184 may be provided to the machine learning model 120 as a training data set to update the machine learning model 120 (e.g., update the machine learning logic 115) based on the feedback 180.

Additionally, or in the alternative, in response to the computing system 110 receiving the feedback 180, the threshold modifier 126 may be configured to update (e.g., modify) the confidence score threshold 140A to an updated confidence score threshold 140B. To illustrate, the confidence score threshold 140A may be elevated in response to the feedback 180 indicating that the medical condition 130, as determined by the machine learning model 120, is not applicable to the non-human patient 166. For example, if the model-based interpretation 136 of the medical images 156 does not match the radiologist interpretation 190 of the medical images 156, the confidence score threshold 140A may be elevated. However, the confidence score threshold 140A may be lowered (or unchanged) in response to the feedback 180 indicating that the medical condition 130, as determined by the machine learning model 120, matches the radiologist-determined medical condition 182.

Although the threshold comparison unit 122 and the threshold modifier 126 are illustrated as independent components of the processor 116, in some implementations, the functions and operations of the threshold comparison unit 122 and the threshold modifier 126 may be performed using the machine learning logic 115. For example, in some implementations, the machine learning model 120 may compare the confidence score 132 to the confidence score threshold 140A. In these implementations, updating the confidence score threshold 140A based on the feedback 180 may include updating the machine learning logic 115.

The techniques described with respect to FIG. 1 may reduce the amount of time necessary for the radiologist 164 to interpret the medical images 156, which in turn may significantly improve the timeliness and efficacy of one or more medical treatments for the non-human patient. For example, instead of the radiologist 164 having to interpret the medical images 156 without assistance from visual aids, the radiologist 164 may use the model-based interpretation 136 of the medical images 156 as a starting point. In particular, the radiologist 164 may simply verify whether the model-based interpretation 136 is applicable and accurate. Using the model-based interpretation 136 as the starting point may enable the radiologist 164 to quickly determine the medical condition 182. In scenarios where the model-based interpretation 136 is not applicable, the radiologist 164 may determine the verified medical condition 182 and provide feedback 180 that is used to improve the machine learning model 120.

In some examples, the analysis of the one or more medical images 156 may benefit from review from the radiologist 164 instead of and/or independent of review by the machine learning model 120. For example, in example embodiments, the machine learning model 120 may be trained to recognize these scenarios and perform actions to more rapidly transmit the medical images 156 directly to the radiologist 164 for interpretation (e.g., without the machine learning model 120 annotating or further analyzing the medical images). To illustrate, in example embodiments, upon receiving the medical images 156, the machine learning model 120 may bypass interpretation of the medical images 156 and initiate immediate transmission of the medical images 156 to the radiologist 164 (e.g., the computing device 104) for interpretation. For example, the machine learning model 120 may be trained to recognize characteristics and/or properties of a medical image 156 that require immediate review and interpretation by the radiologist 164. These characteristics and/or properties may be indicative of a severe and/or urgent medical condition. Thus, in scenarios where it may be clinically required for the radiologist 164 to immediately interpret the medical images 156, the machine learning model 120 may be trained to bypass generation of a model-based interpretation 136 and provide the medical images 156 directly to the radiologist 164 for interpretation.

In some examples, the machine learning model 120 may be trained to recognize these scenarios of severe and/or urgent medical conditions and update one or more factors in the medical condition 130, the confidence score 132, and/or one or more medical indicators 154 associated with the one or more medical images 156 and take one or more responsive actions based thereon. For example, based on determining severe and/or urgent medical conditions in the one or more medical images 156, the machine learning model 120 may adjust one or more weights when calculating and/or otherwise analyzing these factors (e.g., increase the contribution of one or more of medical indicators 154). In other examples, the machine learning model 120 may be trained to annotate one or more records associated with the medical images 156 and/or the medical images 156 themselves based on determining severe and/or urgent medical conditions in the one or more medical images 156.

In some examples, based on determining severe and/or urgent medical conditions 130 in the one or more medical images 156, the machine learning model 120 may generate and transmit instructions that cause a computing device to prioritize analysis and/or review by the radiologist 164 (e.g., by transmitting instructions that cause medical images 156 containing the severe and/or urgent medical conditions to be prioritized in a veterinarian radiologist worklist). For example, based on the medical condition 130, the machine learning model 120 may determine an urgency metric, indicating an urgency of the medical condition 130. Urgent medical conditions, such as traumatic injuries, may have a higher priority than non-urgent medical conditions, such as fleas. The machine learning model 120 may generate a dynamic prioritization indicator for the patient record 150 based on the urgency metric. The dynamic prioritization indicator for the patient record 150 may be compared to dynamic prioritization indicators of other patient records to determine a priority. The veterinarian radiology worklist may be updated by inserting the patient record 150 according to its priority with respect to other patient records in the veterinarian radiology worklist. Once a patient record is inserted into the veterinarian radiology worklist according to its initial priority, the priority of the patient record can dynamically change as time elapses and no action is performed. For example, after a particular period of time, a non-urgent patient record may be reclassified as urgent or critical and may be assigned a higher priority in the veterinarian radiology worklist. Other examples are possible.

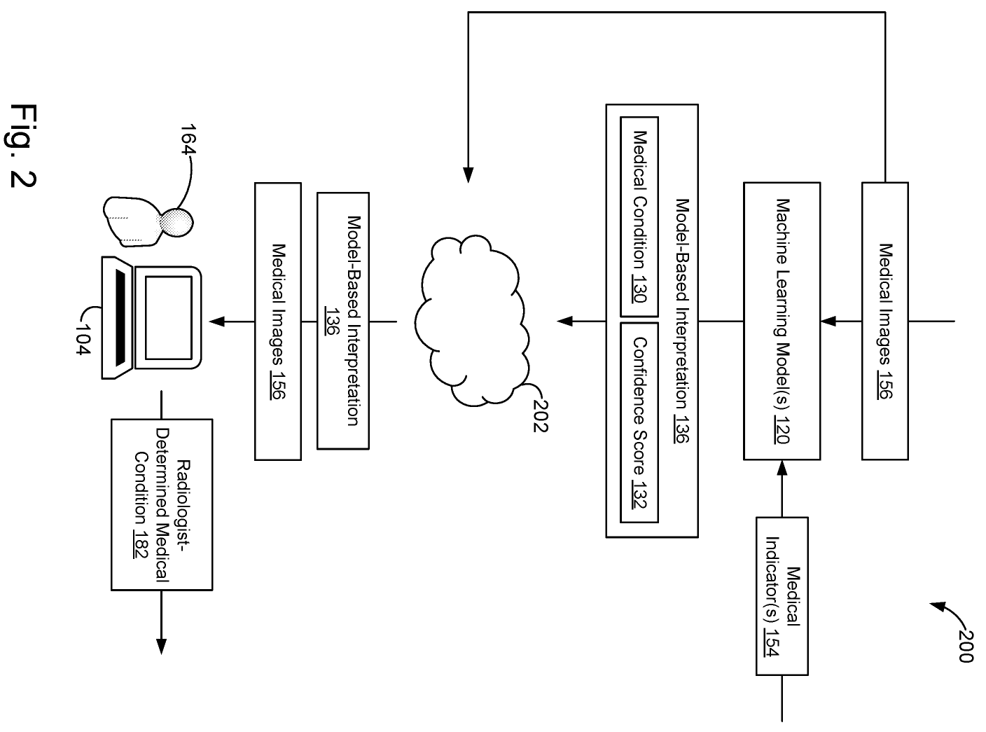

FIG. 2 is a diagram of an example process 200 for interpreting non-human patient medical images using machine learning.

According to the process 200, the medical images 156 of the non-human patient 166 and the medical indicators 154 associated with the non-human patient 166 are provided to the machine learning model 120. Based on the medical images 156 and the medical indicators 154, the machine learning model 120 identifies the medical condition 130 associated with the non-human patient 166. In examples, the identified medical condition 130 may be based on the confidence score 132 that indicates a likelihood that the medical condition 130 is applicable to the non-human patient 166. In examples, the medical condition 130 and the confidence score 132 may be part of the model-based interpretation 136 of the medical images 156.

According to the process 200, the medical images 156 and the model-based interpretation of the medical images 156 are transmitted to the computing device 104 via a network 202. In examples, the radiologist 164 associated with the computing device 104 may use the model-based interpretation 136 of the medical images 156 as a starting point to interpret the medical images 156. For example, the radiologist 164 may simply verify whether the model-based interpretation 136 is applicable and accurate. Using the model-based interpretation 136 as the starting point may enable the radiologist 164 to quickly determine the medical condition 182.

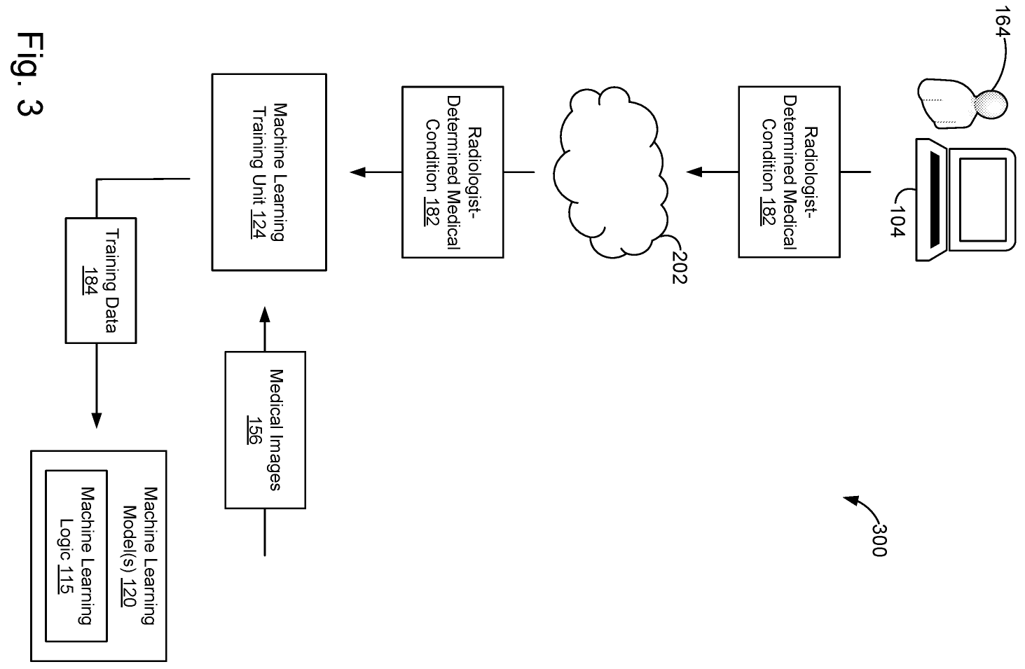

FIG. 3 is a diagram of an example process 300 for training a machine learning model based on a radiologist-determined medical condition. In some examples, the process 300 may be performed in conjunction with the process 200 of FIG. 2.

According to the process 300, the radiologist-determined medical condition 182 is provided to the machine learning training unit 124 via the network 202. Additionally, the medical images 156 are provided to the machine learning training unit 124. In examples, the machine learning training unit 124 generates the training data 184 based on the radiologist-determined medical condition 182 and the medical images 156. In examples, the training data 184 may be used to update the machine learning logic 115. For example, the training data 184 may be provided to the machine learning model 120 as a training data set to update the machine learning model 120 (e.g., update the machine learning logic 115).

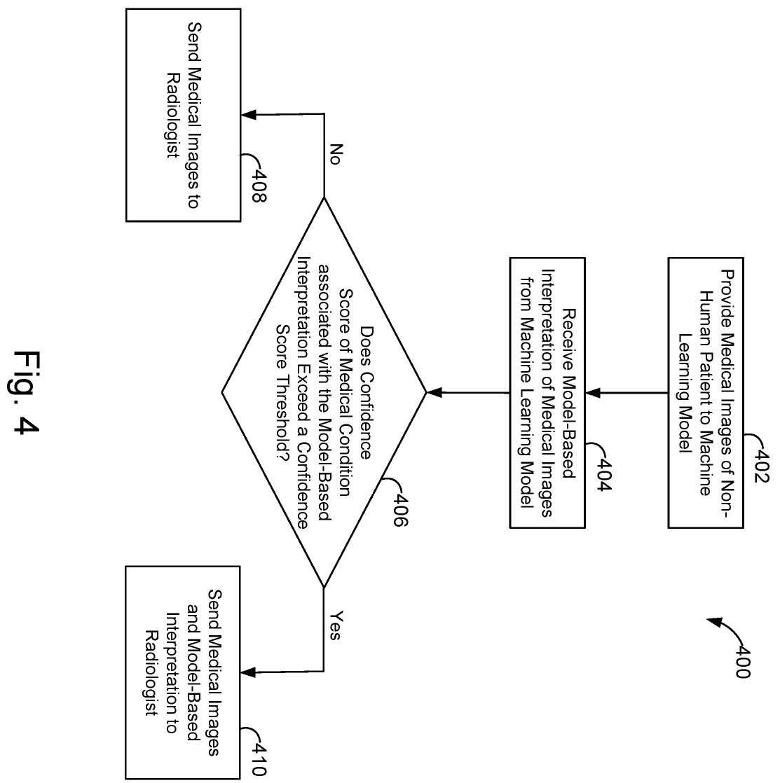

FIG. 4 is a diagram of an example process 400 for selectively sending a model-based interpretation of medical images to a radiologist.

According to the process 400, at step 402, the medical images 156 of the non-human patient 166 may be provided to the machine learning model 120.

At step 404, the computing system 110 may receive the model-based interpretation 136 of the medical images 156 from the machine learning model.

At decision step 406, the computing system 110 and/or the machine learning model 120 may determine whether the confidence score 132 of the medical condition 130, as determined by the machine learning model 120, associated with the model-based interpretation 136 exceeds a confidence score threshold 140A. If the confidence score 132 fails to exceed the confidence score threshold 140A, at decision step 406, the process 400 proceeds to step 408. At step 408, the computing system 110 sends the medical images 156 to the radiologist 164. However, if the confidence score 132 exceeds the confidence score threshold 140A, at decision step 406, the process 400 proceeds to step 410. At step 410, the computing system 110 sends the medical images 156 and the model-based interpretation 136 of the medical images 156 to the radiologist 164.

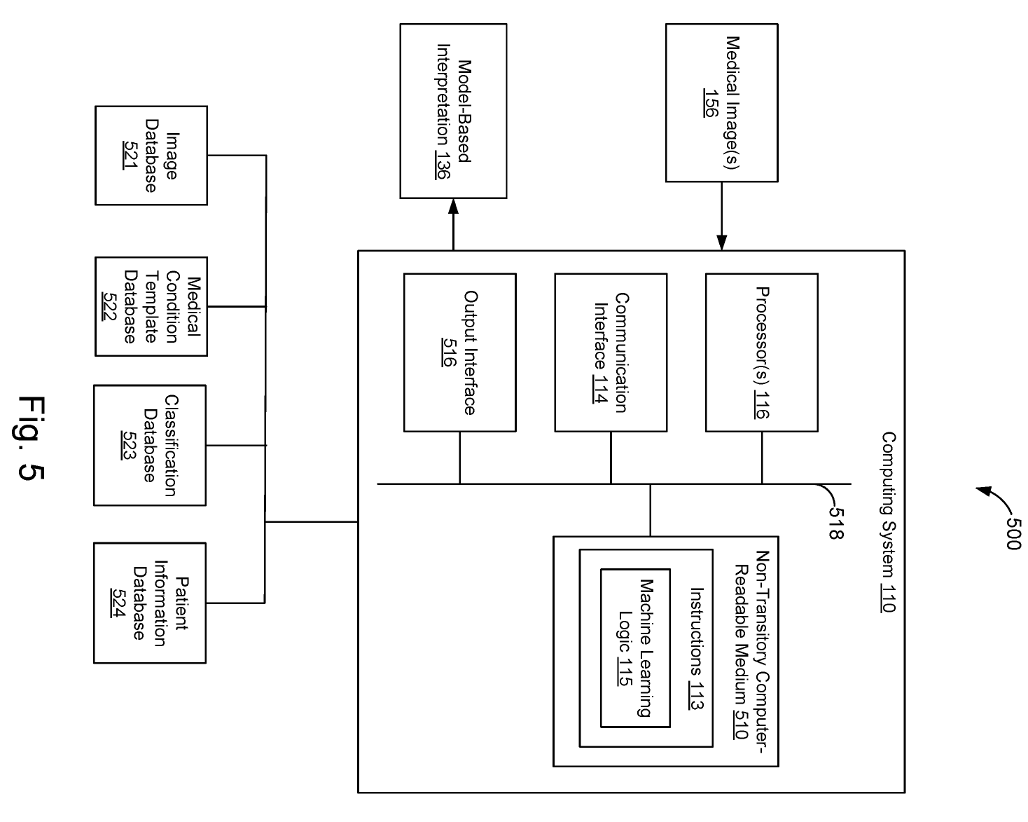

FIG. 5 is a diagram of another example system 500 for interpreting non-human patient medical images using machine learning is depicted. The system 500 includes the computing system 110. The medical images 156 are provided to the computing system 110.

The computing system 110 includes the processor 116 and a non-transitory computer-readable medium 510 (e.g., the memory 112) storing instructions 113 executable by the processor 116 to perform functions for interpreting non-human patient medical images using machine learning. The computing system 110 is shown as a stand-alone component in FIG. 5. However, as indicated above, the computing system 110 may be incorporated within the computing device 102 or the computing device 104.

To perform functions noted above, the computing system 110 also includes the communication interface 114 and an output interface 516, and each component of the computing system 110 is connected to a communication bus 518. In examples, the computing system 110 may also include hardware to enable communication within the computing system 110 and between the computing system 110 and other devices (e.g., the computing devices 102, 104 and/or a server). In examples, the hardware may include transmitters, receivers, and antennas, for example.

In examples, the communication interface 114 may be a wireless interface and/or one or more wireline interfaces that allow for both short-range communication and long-range communication to one or more networks or to one or more remote devices. Such wireless interfaces may provide for communication under one or more wireless communication protocols, Bluetooth, WiFi (e.g., an institute of electrical and electronic engineers (IEEE) 802.11 protocol), Long-Term Evolution (LTE), cellular communications, near-field communication (NFC), and/or other wireless communication protocols. Such wireline interfaces may include an Ethernet interface, a Universal Serial Bus (USB) interface, or similar interface to communicate via a wire, a twisted pair of wires, a coaxial cable, an optical link, a fiber-optic link, or other physical connection to a wireline network. Thus, the communication interface 114 may be configured to receive input data from one or more devices, and may also be configured to send output data to other devices.

In examples, the non-transitory computer readable medium 510 may include or take the form of memory (e.g., the memory 112), such as one or more computer-readable storage media that can be read or accessed by the processor 116. In examples, the non-transitory computer readable medium 510 can include volatile and/or non-volatile storage components, such as optical, magnetic, organic or other memory or disc storage, which can be integrated in whole or in part with the processor 116. In some examples, the non-transitory computer readable medium 510 can be implemented using a single physical device (e.g., one optical, magnetic, organic or other memory or disc storage unit), while in other examples, the non-transitory computer readable medium 510 can be implemented using two or more physical devices. In examples, the non-transitory computer readable medium 510 thus is a computer readable storage, and the instructions 113 are stored thereon. In examples, the instructions 113 include computer executable code.

In examples, the processor 116 may be a general-purpose processor or a special purpose processor (e.g., a digital signal processor, an ASIC, etc.). In examples, the processor 116 may receive inputs from the communication interface 114 (e.g., the medical images 156), and process the inputs to generate outputs that are stored in the non-transitory computer readable medium 510. In examples, the processor 116 can be configured to execute the instructions 113 (e.g., computer-readable program instructions) that are stored in the non-transitory computer readable medium 510 and are executable to provide the functionality of the computing system 110 described herein.

The output interface 516 outputs information for reporting or storage (e.g., the model-based interpretation 136 of the medical images 156), and thus, the output interface 516 may be similar to the communication interface 514 and can be a wireless interface (e.g., transmitter) or a wired interface as well.

In examples, the system 500 can also include or be coupled to a number of databases, such as an image database 521, a medical condition template database 522, a classification database 523, and a patient information database 524. In FIG. 5, the additional databases are shown as separate components of the computing system 110; however, each database may alternatively be integrated within the computing system 110. Access of the databases further enables the computing system 110 to perform functions as described herein. Functionality and content of the databases is described below.

Within one example, in operation, when the instructions 113 are executed by the processor 116, the processor 116 is caused to perform functions including using the machine learning logic 115 to generate the model-based interpretation 136 of the medical images 156.

In examples, the machine learning logic 115 uses statistical models to generate the model-based interpretation 136 of the medical images 156 effectively without using explicit instructions, but instead, can rely on patterns and inferences. In one example, the machine learning logic 115 accesses the image database 521, which includes previously labeled medical images that are indexed using a multi-dimensional indexing scheme based on relevant features/parameters. In such examples, the features/parameters extracted from the medical images 156 under consideration can be compared to the feature data of labeled medical images in the image database 521 to identify particular anatomy or view, and help identify the label of the image captured.

In another example, the machine learning logic 115 can access the medical condition template database 522, which includes templates of different medical conditions constructed using information obtained from the image database 521. For example, feature data over a plurality of known and labeled medical images can be processed using statistical techniques to derive feature data for a template representative over the set of related cases. In this instance, the features/parameters extracted from the medical images 156A can be compared to the feature data for templates in the medical condition template database 522 to identify a particular medical condition.

In another example, the machine learning logic 115 can access the classification database 523, which includes a knowledge base of training data that can be learned from the image database 521 and the template database 522 of previously labeled medical images.

In examples, the machine learning logic 115 can thus operate according to machine learning tasks as classified into several categories. In supervised learning, the machine learning logic 115 builds a mathematical model from a set of data that contains both the inputs and the desired outputs. In examples, the set of data is sample data known as “training data”, in order to make predictions or decisions without being explicitly programmed to perform the task. For example, for determining whether the medical image 156 is an abdomen shot, the training data for a supervised learning algorithm would include images with and without example abdomens for specific species, and each image would have a label (the output) designating whether it contained the abdomen. In examples, the training data for teaching the machine learning logic 115 may be acquired from prior medical image classifications, for example.

In examples, in another category referred to as semi-supervised learning, the machine learning logic 115 can develop mathematical models from incomplete training data, where a portion of the sample input does not have labels. In a further aspect, in examples, a classification algorithm can then be used when the outputs are restricted to a limited set of values.

In examples, in another category referred to as unsupervised learning, the machine learning logic 115 can build a mathematical model from a set of data that contains only inputs and no desired output labels. In a further aspect, in examples, unsupervised learning algorithms are used to find structure in the x-ray images, such as grouping or clustering of data points. In a further aspect, in examples, unsupervised learning can also discover patterns in the x-ray images, and can group the inputs into categories.

In examples, the machine learning logic 115 may be executed to identify anatomy in the medical image 156 and then an appropriate label from a labeling scheme can be applied to the medical image 156. In examples, the type and amount of anatomy that is possible is a finite number, and thus, the machine learning logic 115 may classify the medical images into one of a selected number of groups, such as skull-neck, upper-limb, body-abdomen, lower-limb and other.

Alternative machine learning algorithms 532 may be used to learn and classify the x-ray images, such as deep learning through neural networks or generative models. In examples, deep machine learning may use neural networks to analyze prior medical images through a collection of interconnected processing nodes. In examples, the connections between the nodes may be dynamically weighted. In examples, one or more components of these models (e.g., one or more neural networks) may learn relationships through repeated exposure to data and adjustment of internal weights. In examples, these components (e.g., neural networks) may capture nonlinearity and interactions among independent variables without pre specification. Whereas traditional regression analysis requires that nonlinearities and interactions be detected and specified manually, neural networks perform the tasks automatically.

In examples, a convolutional neural network is a type of neural network. In examples, layers in a convolutional neural network extract features from the medical image 156. In examples, these deep learning methods learn features that are distinctive for classification. For example, convolution neural networks preserve spatial relationship between pixels of images by learning image features using small squares of input data (e.g., filter kernels for convoluting with an input image may be used). In examples, the convolutional neural network is composed for instance of N convolutional layers, M pooling layers, and at least one fully connected layer.

Still other machine learning algorithms or functions can be implemented to identify anatomy of the x-ray images, such as any number of classifiers that receives input parameters and outputs a classification (e.g., attributes of the image). In examples, support vector machine, Bayesian network, a probabilistic boosting tree, neural network, sparse auto-encoding classifier, or other known or later developed machine learning algorithms may be used. However, many other semi-supervised, supervised, or unsupervised learning may be used. In examples, hierarchal, cascade, or other approaches may be also used.

In another example, image processing includes associating patient identification information with the medical images 156. In examples, the computing system 110 can thus access the patient information database 524 to retrieve associated patient identification information. For example, based on the identification of the anatomy in respective medical images 156, the computing system 110 can determine the species of the non-human patient 166, and from the species of the non-human patient 166, the computing system 110 can associate patient identification information with the plurality of medical images 156. In examples, the computing system 110 may further utilize timestamps of the medical images 156 cross referenced with scheduling to access the specific patient identification. In examples, the patient identification information may include species, breed, age, gender, geographical location, or other information as available from the patient information management system (PIMS) that stores information in the patient information database 524. In some examples, however, it may be the case that the patient information is known and already associated with the medical images 156.

In examples, the patient identification information may further be beneficial to assist with the medical image identification. For example, the computing system 110 may be configured to associate patient identification information with the plurality of medical images 156 prior to identification of the medical images, and based on the patient identification information, the computing system 100 further determines the species of the non-human patient 166. Following, the computing system 110 can execute the machine learning logic 115 to select a training data set for use by the machine learning logic 115 based on the species of the non-human patient 166. In examples, the image database 521, the medical condition template database 522, and the classification database 523 all may have different types of training data per different type of species. In examples, the patient identification enables the species to be determined and the correct training data set to be used.

### EXAMPLE METHODS AND ASPECTS

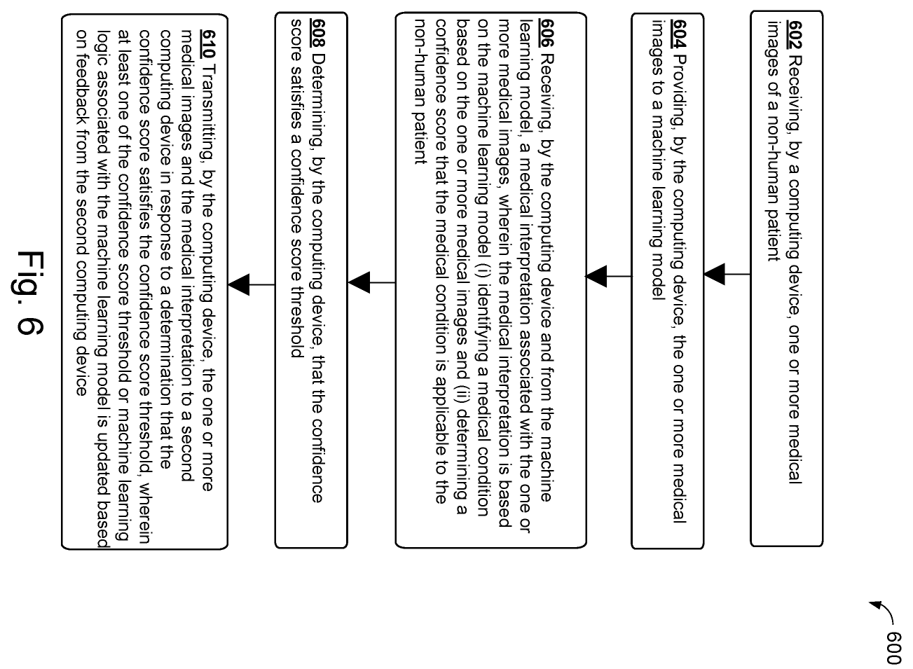

Now referring to FIG. 6, an example method of interpreting non-human patient medical images using machine learning is disclosed.

The method 600 shown in FIG. 6 presents an example of a method that could be used with the components shown in FIGS. 1-5, for example. In other examples, components of the devices and/or systems may be arranged to be adapted to, capable of, or suited for performing the functions, such as when operated in a specific manner. The method 600 may include one or more operations, functions, or actions as illustrated by one or more of blocks 602-610. Although the blocks are illustrated in a sequential order, these blocks may also be performed in parallel, and/or in a different order than those described herein.

Also, the various blocks may be combined into fewer blocks, divided into additional blocks, and/or removed based upon the desired implementation.

At block 602, the method 600 includes receiving, by a computing device, one or more medical images of a non-human patient.

At block 604, the method 600 includes providing, by the computing device, the one or more medical images to a machine learning model.

At block 606, the method 600 includes receiving, by the computing device and from the machine learning model, a medical interpretation associated with the one or more medical images. The medical interpretation is based on the machine learning model (i) identifying a medical condition based on the one or more medical images and (ii) determining a confidence score that the medical condition is applicable to the non-human patient.

At block 608, the method 600 includes determining, by the computing device, that the confidence score satisfies a confidence score threshold.

At block 610, the method 600 includes transmitting, by the computing device, the one or more medical images and the medical interpretation to a second computing device in response to a determination that the confidence score satisfies the confidence score threshold. At least one of the confidence score threshold or machine learning logic associated with the machine learning model is updated based on feedback from the second computing device.

In some examples of the method 600, the feedback indicates whether the medical condition is applicable to the non-human patient. In some examples of the method 600, the confidence score threshold is elevated in response to the feedback indicating that the medical condition is not applicable to the non-human patient. In some examples of the method 600, the confidence score threshold is lowered or unchanged in response to the feedback indicating that the medical condition is applicable to the non-human patient.

In some examples, the method 600 includes generating training data based on the one or more medical images and the feedback. The method 600 may also include updating the machine learning logic based on the training data.

In some examples, the method 600 includes providing one or more medical indicators associated with the non-human patient to the machine learning model. The machine learning model identifies the medical condition and determines the confidence score based on the one or more medical indicators and the one or more medical images.

In some examples of the method 600, the one or more medical indicators associated with the non-human patient comprises one or more of the following: (i) an age of the non-human patient; (ii) a weight of the non-human patient; (iii) a species of the non-human patient; (iv) a breed of the non-human patient; (v) one or more symptoms of the non-human patient; (vi) a preexisting condition of the non-human patient; and (vii) a geographical location of the non-human patient.

In some examples of the method 600, the one or more medical images correspond to radiology images of the non-human patient.

In some examples of the method 600, the second computing device is associated with a radiologist that performs a radiologist reading on the one or more medical images, and wherein the feedback is based on the radiologist reading.

In some examples of the method 600, the machine learning model identifies a species of the non-human patient based on the one or more medical images.

In some examples, the method 600 may include receiving, by the computing device, a particular medical image of a second non-human patient. The method 600 may also include providing, by the computing device, the particular medical image to the machine learning model. The method 600 may also include receiving, by the computing device and from the machine learning model, an indication that the particular medical image requires urgent interpretation. The method 600 may also include transmitting, by the computing device, the particular medical image to the second computing device in response to receiving the indication. In some examples, the machine learning model bypasses performing a medical interpretation on the particular medical image in response to determining that the particular medical image requires urgent interpretation.

In some examples, the method 600 may include determining an urgency metric associated with the medical condition and the confidence score. The method 600 may also include prioritizing a patient record associated with the non-human patient based on the urgency metric. The method 600 may also include generating a veterinarian radiology worklist that includes the patient record. A priority of the patient record in the veterinarian radiology worklist is based the urgency metric.

The method 600 may reduce the amount of time necessary for the radiologist 164 to interpret the medical images 156. For example, instead of the radiologist 164 having to interpret the medical images 156 without assistance from visual aids, the radiologist 164 may use the model-based interpretation 136 of the medical images 156 as a starting point. In particular, the radiologist 164 may simply verify whether the model-based interpretation 136 is applicable and accurate. Using the model-based interpretation 136 as the starting point may enable the radiologist 164 to quickly determine the verified medical condition 182. In scenarios where the model-based interpretation 136 is not applicable, the radiologist 164 may determine the medical condition 182 and provide feedback 180 that is used to improve the machine learning model 120.

It should now be understood that embodiments according to the present disclosure are directed to systems and methods for interpreting non-human patient medical images using machine learning. As outlined above, by interpreting non-human patient medical images using machine learning, radiological assessments may be performed relatively quickly. Without being bound by theory, veterinary patients (e.g., non-human animals) present challenges that are not found in the human health space. For example and in particular, non-human animals are unable to communicate verbally with health care providers and are thus unable to express their medical condition (e.g., what symptoms they are experiencing, where pain exists, how long symptoms have been present, etc.). By contrast human patients are generally able to verbalize their medical condition, symptoms, and the like. As such, radiological assessments in the human space can be based on information gathered directly from the patient. Because non-human patents cannot verbalize their condition, information relevant to radiological assessments is unavailable, and alternate systems and methods that interpret medical images are necessary. Moreover, because embodiments according to the present disclosure interpret medical images using machine learning, the methods according to the present disclosure cannot practically be performed in the human mind.

The singular forms of the articles “a,” “an,” and “the” include plural references unless the context clearly indicates otherwise. For example, the term "a compound" or "at least one compound" can include a plurality of compounds, including mixtures thereof.

Various aspects and embodiments have been disclosed herein, but other aspects and embodiments will certainly be apparent to those skilled in the art. Additionally, the various aspects and embodiments disclosed herein are provided for explanatory purposes and are not intended to be limiting, with the true scope being indicated by the following claims.

What is claimed is:

1. A computer-implemented method for interpreting non-human patient medical images, the computer-implemented method comprising:
receiving, by a computing device, one or more medical images of a non-human patient;
providing, by the computing device, the one or more medical images to a machine learning model;
receiving, by the computing device and from the machine learning model, a medical interpretation associated with the one or more medical images, wherein the medical interpretation is based on the machine learning model:
identifying a medical condition based on the one or more medical images; and
determining a confidence score that the medical condition is applicable to the non-human patient;
determining, by the computing device, that the confidence score satisfies a confidence score threshold; and
transmitting, by the computing device, the one or more medical images and the medical interpretation to a second computing device in response to a determination that the confidence score satisfies the confidence score threshold, wherein at least one of the confidence score threshold or machine learning logic associated with the machine learning model is updated based on feedback from the second computing device.

2. The computer-implemented method of claim 1, wherein the feedback indicates whether the medical condition is applicable to the non-human patient.

3. The computer-implemented method of claim 2, wherein the confidence score threshold is elevated in response to the feedback indicating that the medical condition is not applicable to the non-human patient.

4. The computer-implemented method of claim 2, wherein the confidence score threshold is lowered or unchanged in response to the feedback indicating that the medical condition is applicable to the non-human patient.

5. The computer-implemented method of claim 2, further comprising:
generating training data based on the one or more medical images and the feedback; and
updating the machine learning logic based on the training data.

6. The computer-implemented method of claim 1, further comprising providing one or more medical indicators associated with the non-human patient to the machine learning model, wherein the machine learning model identifies the medical condition and determines the confidence score based on the one or more medical indicators and the one or more medical images.

7. The computer-implemented method of claim 6, wherein the one or more medical indicators associated with the non-human patient comprises one or more of the following: (i) an age of the non-human patient; (ii) a weight of the non-human patient; (iii) a species of the non-human patient; (iv) a breed of the non-human patient; (v) one or more symptoms of the non-human patient; (vi) a preexisting condition of the non-human patient; and (vii) a geographical location of the non-human patient.

8. The computer-implemented method of claim 1, wherein the one or more medical images correspond to radiology images of the non-human patient.

9. The computer-implemented method of claim 1, wherein the second computing device is associated with a radiologist that performs a radiologist reading on the one or more medical images, and wherein the feedback is based on the radiologist reading.

10. The computer-implemented method of claim 1, wherein the machine learning model identifies a species of the non-human patient based on the one or more medical images.

11. The computer-implemented method of claim 1, further comprising:
receiving, by the computing device, a particular medical image of a second non-human patient;
providing, by the computing device, the particular medical image to the machine learning model;
receiving, by the computing device and from the machine learning model, an indication that the particular medical image requires urgent interpretation; and
transmitting, by the computing device, the particular medical image to the second computing device in response to receiving the indication.

12. The computer-implemented method of claim 11, wherein the machine learning model bypasses performing a medical interpretation on the particular medical image in response to determining that the particular medical image requires urgent interpretation.

13. The computer-implemented method of claim 1, further comprising:
determining an urgency metric associated with the medical condition and the confidence score; and
generating a veterinarian radiology worklist that includes a patient record associated with the non-human patient, wherein a priority of the patient record in the veterinarian radiology worklist is based the urgency metric.

14. A computing device comprising:
one or more processors; and
a tangible, non-transitory computer-readable medium comprising instructions that, when executed by the one or more processors, cause the one or more processors to perform operations comprising:
receiving one or more medical images of a non-human patient;
providing the one or more medical images to a machine learning model;
receiving, from the machine learning model, a medical interpretation associated with the one or more medical images, wherein the medical interpretation is based on the machine learning model:
identifying a medical condition based on the one or more medical images; and
determining a confidence score that the medical condition is applicable to the non-human patient;
determining that the confidence score satisfies a confidence score threshold; and
transmitting the one or more medical images and the medical interpretation to a second computing device in response to a determination that the confidence score satisfies the confidence score threshold, wherein at least one of the confidence score threshold or machine learning logic associated with the machine learning model is updated based on feedback from the second computing device.

15. The computing device of claim 14, wherein the feedback indicates whether the medical condition is applicable to the non-human patient.

16. The computing device of claim 15, wherein the confidence score threshold is elevated in response to the feedback indicating that the medical condition is not applicable to the non-human patient.

17. The computing device of claim 15, wherein the confidence score threshold is lowered or unchanged in response to the feedback indicating that the medical condition is applicable to the non-human patient.

18. The computing device of claim 15, wherein the operations further comprise:
generating training data based on the one or more medical images and the feedback; and
updating the machine learning logic based on the training data.

19. The computing device of claim 14, wherein the operations further comprise providing one or more medical indicators associated with the non-human patient to the machine learning model, wherein the machine learning model identifies the medical condition and determines the confidence score based on the one or more medical indicators and the one or more medical images.

20. The computing device of claim 19, wherein the one or more medical indicators associated with the non-human patient comprises one or more of the following: (i) an age of the non-human patient; (ii) a weight of the non-human patient; (iii) a species of the non-human patient; (iv) a breed of the non-human patient; (v) one or more symptoms of the non-human patient; (vi) a preexisting condition of the non-human patient; and (vii) a geographical location of the non-human patient.

21. The computing device of claim 14, wherein the one or more medical images correspond to radiology images of the non-human patient.

22. The computing device of claim 14, wherein the second computing device is associated with a radiologist that performs a radiologist reading on the one or more medical images, and wherein the feedback is based on the radiologist reading.

23. A tangible, non-transitory computer-readable medium comprising instructions that, when executed by one or more processors of a computing device, cause the one or more processors to perform operations comprising:
receiving one or more medical images of a non-human patient;
providing the one or more medical images to a machine learning model;
receiving, from the machine learning model, a medical interpretation associated with the one or more medical images, wherein the medical interpretation is based on the machine learning model:
identifying a medical condition based on the one or more medical images; and
determining a confidence score that the medical condition is applicable to the non-human patient;
determining that the confidence score satisfies a confidence score threshold; and
transmitting the one or more medical images and the medical interpretation to a second computing device in response to a determination that the confidence score satisfies the confidence score threshold, wherein at least one of the confidence score threshold or machine learning logic associated with the machine learning model is updated based on feedback from the second computing device.
